SEQUENCE LISTING (7 sequences)

SEQ ID NO: 1 - DNA, 101 bp, synthetic construct
  misc_feature 1..101: note = Synthetic primer
  source 1..101: mol_type = other DNA
ctctcggcatggacgagctgtacaaggctactaacttcagcctgctgaagcaggctggagacgtggaggagaaccctggacctgtcgttttacaacgtcgt

SEQ ID NO: 2 - DNA, 75 bp, synthetic construct
  misc_feature 1..75: note = Synthetic primer
  source 1..75: mol_type = other DNA
gggtcctggattttcctcaacatctccacaagtaagcaaagagccccttccttctttttgacaccagaccaactg

SEQ ID NO: 3 - DNA, 40 bp, synthetic construct
  misc_feature 1..40: note = Synthetic Primer
  source 1..40: mol_type = other DNA
ttgaggaaaatccaggacccatggaagacgccaaaaacat

SEQ ID NO: 4 - DNA, 41 bp, synthetic construct
  misc_feature 1..41: note = Synthetic primer
  source 1..41: mol_type = other DNA
agagtcgcggccgctttacttttacaatttggactttccgc

SEQ ID NO: 5 - DNA, 9161 bp, synthetic construct
  misc_feature 1..9161: note = Reporter gene expression cassette
  misc_feature 1..314: note = 5'ITR
  regulatory 408..1997: note = promoter - CAG promoter; regulatory_class = promoter
  misc_feature 2051..2084: note = loxP sequence
  regulatory 2085..2908: note = terminator - STOP sequence; regulatory_class = terminator
  misc_feature 2909..2942: note = loxP sequence
  gene 3031..3747: note = EGFP
  misc_feature 3748..3804: note = P2A peptide
  gene 3805..6849: note = LacZ
  misc_feature 6850..6903: note = T2A peptide
  gene 6904..8555: note = luciferase
  misc_feature 8920..9161: note = 3'ITR
  source 1..9161: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaactagaatgctagaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggcctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattataacttcgtatagcatacattatacgaagttatagcttatcgatatttaaatcctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaaccggataacttcgtatagcatacattatacgaagttatgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggctactaacttcagcctgctgaagcaggctggagacgtggaggagaaccctggacctgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaagaaggaaggggctctttgcttacttgtggagatgttgaggaaaatccaggacccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaattcatgtcgacatactagttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 6 - DNA, 2460 bp, synthetic construct
  misc_feature 1..2460: note = Selectable markar gene expression cassette
  misc_feature 1..314: note = 5'ITR
  regulatory 584..1105: note = promoter - PKG promoter; regulatory_class = promoter
  gene 1106..1909: note = Neo
  misc_feature 2219..2460: note = 3'ITR
  source 1..2460: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaactagaatgctagaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcataaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatcctagttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 7 - DNA, 3499 bp, synthetic construct
  misc_feature 1..3499: note = Transposasegene expression cassette
  regulatory 1..1667: note = promoter - CAG promoter; regulatory_class = promoter
  gene 1721..3499: note = Hyper Piggybase
  source 1..3499: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattatgggcagcagcctggacgacgagcacatcctgagcgccctgctgcagagcgacgacgagctggtcggcgaggacagcgacagcgaggtgagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagcccaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgacctccgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcagggtctatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgggcctgacctccagcttcatgaggaagaggctggaggcccccaccctgaagagatacctgagggacaacatcagcaacatcctgcccaaagaggtgcccggcaccagcgacgacagcaccgaggagcccgtgatgaagaagaggacctactgcacctactgtcccagcaagatcagaagaaaggccagcgccagctgcaagaagtgtaagaaggtcatctgccgggagcacaacatcgacatgtgccagagctgt

(19) United States
(12) Patent Application Publication
(10) Pub. No.: US 2024/0344153 A1
(43) Pub. Date: Oct. 17, 2024

(54) METHOD FOR EVALUATING VIRUS INFECTION

(71) Applicant: The University of Tokyo, Tokyo (JP)

(72) Inventors: Takashi OKADA, Tokyo (JP); Yuji TSUNEKAWA, Tokyo (JP)

(73) Assignee: The University of Tokyo, Tokyo (JP)

(21) Appl. No.: 18/700,038

(22) PCT Filed: Oct. 12, 2022
(86) PCT No.: PCT/JP2022/038051

§ 371 Date: Apr. 10, 2024

(30) Foreign Application Priority Data
Oct. 13, 2021 (JP) 2021-167826

(51) Int. Cl. C12Q 1/6897; C12N 15/85

(52) U.S. Cl. CPC C12Q 1/6897; C12N 15/85

(57) ABSTRACT

It is an object of the present invention to provide a method of detecting an AAV (adeno-associated virus)-neutralizing antibody with high sensitivity, and tools used in this method (e.g. DNA, cells, etc.). Specifically, it is the object of the present invention to provide a vector composition for detection of an AAV (adeno-associated virus)-neutralizing antibody, the vector composition comprising the following vectors (a), (b) and (c):
(a) a vector, comprising a DNA in which a 5′ transposon-specific inverted terminal repeat (ITR) sequence (hereinafter referred to as a “5′ ITR sequence”), a promoter, a transcription termination STOP sequence that can be removed by a site-specific recombinant enzyme, one or multiple reporter genes, and a 3′ transposon ITR sequence (hereinafter referred to as a “3′ ITR sequence”) are connected with one another in this order, the DNA being disposed such that if the transcription termination STOP sequence is removed, the one or multiple reporter genes can be expressed by the promoter (hereinafter referred to as a “reporter gene expression cassette”),
(b) a vector, comprising a DNA in which a 5′ ITR sequence, a promoter, a selection marker gene, and a 3′ ITR sequence are connected with one another in this order, the DNA being disposed such that the selection marker gene can be expressed by the promoter (hereinafter referred to as a “selection marker gene expression cassette”), and
(c) a vector, comprising a DNA in which a promoter and a transposase gene that recognizes the transposon ITR sequences included in the vectors (a) and (b) are connected with each other in this order (hereinafter referred to as a “transposase gene expression cassette”), the DNA being disposed such that the transposase gene can be expressed by the promoter.

### TECHNICAL FIELD

The present invention relates to a method for evaluating the infection efficiency of a virus used as a vector for gene therapy. More specifically, the present invention relates to a method of measuring the amount of a neutralizing antibody against an adeno-associated virus (AAV).

### BACKGROUND ART

When the adaptability of gene therapy for a patient is determined upon implementation of the gene therapy, it is useful to evaluate the infection efficiency of a virus used as a vector, specifically, to measure the amount of a neutralizing antibody against the virus possessed by the patient. In recent years, as the application of gene therapy using AAV vectors has progressed, adverse events associated with systemic administration of large amounts of the AAV vectors have frequently occurred, and thus, in order to ensure the safety and efficacy of the AAV vectors, the importance of evaluation of the neutralizing antibody is increasing.

It has been found that when a patient has a neutralizing antibody against AAV (i.e., an antibody that inhibits infection of AAV into cells; hereinafter referred to as an “AAV-neutralizing antibody”), the AAV-neutralizing antibody binds to an AAV vector administered to the patient and inhibits infection of the AAV into cells (Non Patent Literature 1), so that desired therapeutic effects cannot be obtained. In fact, if a subject is determined to be positive in the evaluation of the presence or absence of an AAV-neutralizing antibody in the subject, the subject is excluded from enrollment in clinical trials using AAV vectors. Therefore, accurate detection of an AAV-neutralizing antibody is important to evaluate the suitability of gene therapy using AAV vectors.

However, the methods of measuring AAV-neutralizing antibodies differ among various clinical testing laboratories, and are not standardized at present. Currently, there are two main methods of detecting an anti-AAV antibody in vitro. One is an ELISA-based method, and the other is a cell-based method involving AAV transduction.

The ELISA-based method is easy to set up, etc. and can be carried out relatively simply. However, for example, the ELISA method using antibodies against AAV-neutralizing antibodies has a high degree of cross reactivity between AAV-neutralizing antibodies of different serotypes, and also has insufficient sensitivity, resulting in easy generation of false positives and false negatives. Accordingly, it is difficult to appropriately select applicable cases.

On the other hand, the cell-based method is a method of determining the presence or absence, abundance, neutralizing activity, etc. of an AAV-neutralizing antibody, the method comprising incubating subject-derived serum and an AAV vector, and then evaluating the efficiency of transduction of the AAV vector into cells (Non Patent Literature 2 and Non Patent Literature 3). Compared with the ELISA-based method, the cell-based method is superior in that the presence or absence of inhibition of AAV vector transduction can be directly detected, and it has been reported that the cell-based method has been improved also in terms of low detection sensitivity, which has been a problem in the past (Non Patent Literature 4).

As described above, the conventional methods of measuring the amount of an AAV-neutralizing antibody, in particular, the cell-based method, has been improved in terms of sensitivity, but there is still room for further improvement, such as standardization of the measurement method.

### CITATION LIST

### Non Patent Literature

Non Patent Literature 1: Wang et al., Nature Reviews Drug Discovery 18:358-378, 2019.

Non Patent Literature 2: Kruzik et al., Human Gene Therapy Methods 30:35-43, 2019.

Non Patent Literature 3: Meliani et al., Human Gene Therapy Methods 26:45-53, 2015.

Non Patent Literature 4: Baatartsogt et al., Molecular Therapy Methods & Clinical development 22:162-171, 2021.

### SUMMARY OF INVENTION

### Technical Problem

Considering the aforementioned circumstances, it is an object of the present invention to provide a method of detecting an AAV-neutralizing antibody with high sensitivity, and tools used in this method (e.g. DNA, cells, etc.).

### Solution to Problem

Utilizing the ITR sequence of a transposon, the present inventors have produced cells, in which many copies of the reporter genes are inserted into the genome thereof so that the genes can be expressed, and have developed a system of detecting AAV infection with high sensitivity, using the produced cells. By using the AAV infection detection system developed by the present inventors, it becomes possible to detect an AAV-neutralizing antibody in a sample with high sensitivity.

Specifically, the present invention includes the following (1) to (8).
(1) A vector composition for detection of an AAV (adeno-associated virus)-neutralizing antibody, the vector composition comprising the following vectors (a), (b) and (c):
   (a) a vector, comprising a DNA in which a 5′ transposon-specific inverted terminal repeat (ITR) sequence (hereinafter referred to as a “5′ITR sequence”), a promoter, a transcription termination STOP sequence that can be removed by a site-specific recombinant enzyme, one or multiple reporter genes, and a 3′ transposon ITR sequence (hereinafter referred to as a “3′ ITR sequence”) are connected with one another in this order, the DNA being disposed such that if the transcription termination STOP sequence is removed, the one or multiple reporter genes can be expressed by the promoter (hereinafter referred to as a “reporter gene expression cassette”),
   (b) a vector, comprising a DNA in which a 5′ ITR sequence, a promoter, a selection marker gene, and a 3′ ITR sequence are connected with one another in this order, the DNA being disposed such that the selection marker gene can be expressed by the promoter (hereinafter referred to as a “selection marker gene expression cassette”), and
   (c) a vector, comprising a DNA in which a promoter and a transposase gene that recognizes the transposon ITR sequences included in the vectors (a) and (b) are connected with each other in this order (hereinafter referred to as a “transposase gene expression cassette”), the DNA being disposed such that the transposase gene can be expressed by the promoter.
(2) The vector composition according to the above (1), which is characterized in that the STOP sequence is disposed between site-specific recombinant enzyme-recognizing sequences disposed in the same direction.
(3) The vector composition according to the above (2), wherein the site-specific recombinant enzyme-recognizing sequences are loxP sequences, and the transposon is piggy Bac.
(4) A cell, in which the reporter gene expression cassette, the selection marker gene expression cassette, and the transposase gene expression cassette according to any one of the above (1) to (3) are inserted into the genome thereof.
(5) A method of detecting an AAV-neutralizing antibody in a sample, comprising:
   a step of adding a mixture of recombinant AAV retaining a site-specific recombinant enzyme gene in an expressible manner and a sample to the cell according to claim 4, so that the AVV is infected into the cell, and
   a step of measuring the expression level of a reporter gene.
(6) The method according to the above (5), wherein the site-specific recombinant enzyme is Cre recombinase.
(7) An AAV-neutralizing antibody detection kit, including at least one vector among a vector comprising a reporter gene expression cassette, a vector comprising a selection marker gene expression cassette, and a vector comprising a transposase gene expression cassette.
(8) An AAV-neutralizing antibody detection kit, including the cell according to the above (4).

It is to be noted that the preposition “to” used in the present description indicates a numerical value range including the numerical values located left and right of the preposition.

### Advantageous Effects of Invention

By the present invention, a system of detecting AAV infection into cells with high sensitivity is provided. By using this system, it becomes possible to evaluate the abundance of an AAV-neutralizing antibody in a sample with high sensitivity and good reproducibility.

According to the present invention, it is possible to provide highly sensitive receiver cells (cells to be infected with AAV, which are used for the assay of an AAV-neutralizing antibody). As a result, it becomes possible to stably detect an AAV-neutralizing antibody with high sensitivity.

### BRIEF DESCRIPTION OF DRAWINGS

FIG. 1 shows a schematic view of the configuration of a reporter gene expression cassette, a selection marker gene expression cassette and a transposase gene expression cassette, which are comprised in the vector according to the present invention.

FIG. 2 shows the results obtained by confirming the expression of reporter genes, using the cells according to the present invention that are for use in highly sensitive detection of an AAV-neutralizing antibody. Regarding the expression state of the reporter genes in the cells obtained as a result of screening, FIG. 2A shows the results of confirmation of GFP activity, and FIG. 2B shows the results of confirmation of luciferase activity.

FIG. 3 shows the results obtained by measuring luciferase activity in T137-30 cells selected based on the results shown in FIG. 2. These results show that self-replicating Cre packaging AAV1 (SC-Cre-NaB), single-stranded Cre packaging AAV1 (SS-Cre-Nab), or single-stranded luciferase packaging AAV1 (Ss-Luc-Nab) was successively diluted 10-fold and was then each transfected into the T137-30 cells, and the luciferase activity was then measured.

### DESCRIPTION OF EMBODIMENTS

Hereafter, the embodiments for carrying out the present invention will be described.

A first embodiment relates to a vector composition for detection of an AAV-neutralizing antibody, the vector composition comprising the following vectors (a), (b) and (c) (hereinafter also referred to as “the vector composition according to the present embodiment”):
(a) a vector (hereinafter referred to as a “reporter gene expression vector”), comprising a DNA in which a 5′ transposon-specific inverted terminal repeat (ITR) sequence (hereinafter referred to as a “5′ ITR sequence”), a promoter, a transcription termination STOP sequence that can be removed by a site-specific recombinant enzyme, one or multiple reporter genes, and a 3′ transposon ITR sequence (hereinafter referred to as a “3′ ITR sequence”) are connected with one another in this order, the DNA being disposed such that if the transcription termination STOP sequence is removed, the one or multiple reporter genes can be expressed by the promoter (hereinafter referred to as a “reporter gene expression cassette”),
(b) a vector (hereinafter referred to as a “selection marker expression vector”), comprising a DNA in which a 5′ ITR sequence, a promoter, a selection marker gene, and a 3′ ITR sequence are connected with one another in this order, the DNA being disposed such that the selection marker gene can be expressed by the promoter (hereinafter referred to as a “selection marker gene expression cassette”), and
(c) a vector (hereinafter referred to as a “transposase expression vector”), comprising a DNA in which a promoter and a transposase gene that recognizes the transposon ITR sequences included in the vectors (a) and (b) are connected with each other in this order (hereinafter referred to as a “transposase gene expression cassette”), the DNA being disposed such that the transposase gene can be expressed by the promoter.

In the present embodiment, the term “transposon” is used to mean a nucleic acid sequence that transposes to a different position on the DNA in a cell, and in the present embodiment, it particularly means a DNA transposon (class II transposon). At both termini of the DNA transposon, a 5′ ITR sequence and a 3′ ITR sequence specific to each transposon are present. The transposase specific to each transposon recognizes the ITR sequences that are present at both termini, excises the transposon DNA from the genome, and inserts the excised transposon DNA into another position on the genome. Utilizing the functions of such transposon and transposase, the position on the genome of DNA inserted between the two ITR sequences can be changed. Examples of the transposon that can be used in the present embodiment may include, but are not particularly limited to, piggy Bac, Sleeping Beauty, Tol2, and P element (Ivics et al., Nat Methods. 6:415-422, 2009.).

In the present embodiment, the term “site-specific recombination” is used to mean a phenomenon in which DNA recombination occurs between specific homologous nucleotide sequences (site-specific recombinant sequences). The site-specific recombination is induced by a site-specific recombinant enzyme specific to the homologous nucleotide sequences. As a system for inducing site-specific recombination by such a site-specific recombinant enzyme, for example, a phage-derived Cre/loxP system is often used. Cre recombinase is a P1 phage-derived DNA recombinase that recognizes a 34-bp site-specific recombinant sequence called a loxP site and induces site-specific recombination. In addition to conventional loxP sequences, mutant loxP sequences such as lox511, lox2272 and loxFAS are also present. The Cre/loxP system is widely utilized for modification of the genetic structure on the genomic DNA, such as deletion, substitution, inversion, etc. of a DNA region between two loxP sequences or mutant loxP sequences. When the two loxP sequences are disposed in the same direction, the DNA between the loxP sequences is cut out by Cre recombinase and is circularized. On the other hand, when the two loxP sequences are disposed in the reverse direction to each other, the direction of the DNA between the loxP sequences is inverted by the action of Cre recombinase

In addition to the Cre/loxP system, other examples of the site-specific recombination system may include an Flp/FRT system derived from the yeast plasmid 2 μ, a Dre/rox system derived from the enterobacteriophage D6, an R/RS system derived from soy sauce yeast, and these site-specific recombination systems can also be used in the present embodiment. In the present embodiment, when the site-specific recombination system is used, the site-specific recombinant sequences are disposed in the same direction in the reporter gene expression cassette.

In the present embodiment, the term “transcription termination STOP sequence” is used to mean a sequence comprising a stop codon and a poly-A addition signal. The transcription termination STOP sequence is not particularly limited, as long as it exhibits the effect of transcription termination, and a part of DNA included in a commercially available vector, etc. can also be used. In addition, the promoter is not particularly limited, and persons skilled in the present technical field can appropriately select the promoter. Examples of the promoter may include a CAG promoter and a PKG promoter.

In the present embodiment, the term “selection marker gene” is used to mean a gene that imparts a selectable phenotype to cells into which the selection marker gene has been introduced. Those skilled in the art can easily select a selection marker suitable for the present embodiment. The suitable selection marker gene is not particularly limited, and examples of the selection marker gene may include genes that have influence on cell proliferation, such as a Neo gene (selected with G418), a Hyg gene (selected with hygromycin), a hisD gene (selected with histidinol), a Gpt gene (selected with 6-thioxanthin), and Ble gene (selected by bleomycin).

The reporter gene used in the present embodiment is not particularly limited, and examples of the reporter gene used herein may include genes encoding fluorescent proteins such as EGFP and mCherry, and genes encoding luciferase and β-galactosidase. The reporter gene expression cassette according to the present embodiment may comprise one or multiple reporter genes. When the present reporter gene expression cassette comprises multiple reporter genes, it is desirable that the multiple reporter genes are comprised in the reporter gene expression cassette in such an arrangement that the multiple reporter genes are expressed (polycistronic expression). In order to enable the polycistronic expression of the reporter genes, for example, a DNA sequence encoding a 2A self-cleaving peptide may be inserted between individual reporter genes. If the DNA encoding the 2A peptide is inserted, for example, between two reporter genes, during translation of these reporter genes, the translated 2A peptide inhibits the peptide transferase activity of the ribosome, and the linkage of the polypeptide chains is prevented (“ribosomal skipping”). As a result of such ribosomal skipping, all of individual reporter genes are translated into proteins. The coding sequence of the 2A peptide used in the present embodiment is not particularly limited, and examples thereof may include sequences encoding T2A, P2A, E2A and F2A, etc. (Ziqing et al., Sci Rep. 7:2193. doi: 10.1038/s41598-017-02460-2, 2017).

A second embodiment relates to a cell in which the reporter gene expression cassette, selection marker gene expression cassette, and transposase gene expression cassette according to the first embodiment are inserted into the genome thereof (the cell according to the second embodiment: also referred to as a “receiver cell” in the present description).

The cell according to the second embodiment can be obtained by transfecting the vector composition for detection of an AAV-neutralizing antibody according to the first embodiment (the vector composition according to the present embodiment) into cells, and performing selection with a selection marker. Herein, the composition ratio (molar ratio) of the reporter gene expression vector, the selection marker gene expression vector and the transposase expression vector comprised in the vector composition according to the present embodiment is not particularly limited, and for example, setting the composition ratio of the selection marker gene expression vector as 1, the reporter gene expression vector is 5 to 35, preferably 10 to 30, and more preferably 15 to 25, and the transposase gene expression vector is 1 to 5.

The reporter gene expression vector, selection marker gene expression vector and transposase gene expression vector according to the present embodiment can be produced by constructing each expression cassette on a known vector based on the ordinary techniques in the present technical field. The known vectors used herein are not particularly limited, and examples thereof may include a pUC-based plasmid and a pBR322-based plasmid, which can be amplified using E. coli.

The cell according to the second embodiment may be prepared using, for example, HEK293 cells, CHO cells, 3T3 cells, and the like, but are not particularly limited thereto.

A third embodiment relates to a method of detecting an AAV-neutralizing antibody.

More specifically, this is a method of detecting an AAV-neutralizing antibody existing in a sample (for example, a blood-derived sample (serum, etc.)), the method comprising:
a step of adding a mixture of recombinant AAV retaining a site-specific recombinant enzyme gene in an expressible manner (hereinafter also referred to as “recombinant enzyme-expressing AAV”) and a sample to the cell according to the second embodiment, so that the AVV is infected into the cell, and
a step of measuring the expression level of a reporter gene.

The recombinant enzyme-expressing AAV is AAV that retains a site-specific recombinant enzyme (e.g., Cre recombinase (Cre), flippase (Flp), Dre recombinase (Dre), R enzyme (R), etc.) gene and a promoter that regulates the expression of the gene.

In the cell according to the second embodiment, a reporter gene expression cassette, a selection marker gene expression cassette, and a transposase gene expression cassette are inserted into the genome thereof, and a selection marker gene and a transposase are in a state in which they can be expressed by a promoter (i.e., the selection marker gene and the transposase gene are disposed in each expression cassette so that each of them can be expressed by the promoter that is present on the 5′ side). Since a transcription termination STOP sequence is present between the promoter and a reporter gene, the reporter gene cannot be expressed as it stands in the current state.

When the recombinant enzyme-expressing AAV is infected into the cell of the second embodiment and the site-specific recombinant enzyme is then expressed in the cell, homologous recombination is induced between two site-specific recombinant sequences (e.g., loxP sequences, etc.) that are inserted into the genome of the cell in the same direction by the function of this enzyme, and the transcription termination STOP sequence is removed from the cell genome. As a result, the reporter gene on the genome becomes a state in which it can be expressed by regulation of the promoter in the reporter gene expression cassette.

Therefore, when the recombinant enzyme-expressing AAV is infected into the cell of the second embodiment, the reporter gene becomes expressible, and thus, reporter signals (e.g., fluorescent signals, luminescent signals or enzymatic activities, etc.) can be detected. By detecting these reporter signals, infection of the recombinant enzyme-expressing AAV into the cell can be detected.

When an AAV-neutralizing antibody is present at a stage in which the recombinant enzyme-expressing AAV is infected into the cell, infection of the AAV into the cell is suppressed, and the expression of the site-specific recombinant enzyme in the cell is also suppressed. As a result, the reporter signals are also decreased. The decrease in the reporter signals can be used as an indicator to detect the presence or absence of an AAV-neutralizing antibody in a sample and to evaluate the abundance of the AAV-neutralizing antibody in the sample.

The recombinant enzyme-expressing AAV used in the third embodiment may be any AAV, as long as it retains the site-specific recombinant enzyme in an expressible manner, and may be of any serotype (AAV1, AAV2, AAV3, AAV4, AAV5, AAV6, AAV7, AAV8, and AAV9). For example, if the serotype of the recombinant enzyme-expressing AAV is AAV1, it is possible to detect a neutralizing antibody against AAV1 in the sample and to evaluate its abundance. The same is true for other serotypes.

A fourth embodiment relates to an AAV-neutralizing antibody detection kit, including at least one vector among a vector comprising a reporter gene expression cassette, a vector comprising a selection marker gene expression cassette, and a vector comprising a transposase gene expression cassette. In addition, the AAV-neutralizing antibody detection kit according to the fourth embodiment may include the cell according to the second embodiment. Furthermore, there is a case where the present kit further includes one or multiple additional reagents. Examples of such additional reagents may include a dilution buffer, a reconstitution solution, a washing buffer, a nucleic acid-introducing reagent, a protein-introducing reagent, and a control reagent, but the examples are not limited thereto. In general, an instruction manual is included with the kit.

When the present description is translated into English and the English description includes singular terms with the articles “a,” “an,” and “the,” these terms include not only single items but also multiple items, unless otherwise clearly specified from the context that it is not the case.

Hereinafter, the present invention will be further described in the following examples. However, these examples are only illustrative examples of the embodiments of the present invention, and thus, are not intended to limit the scope of the present invention.

### EXAMPLES

### 1. Production of Various Types of Vectors

### 1-1. Reporter Gene Expression Vector

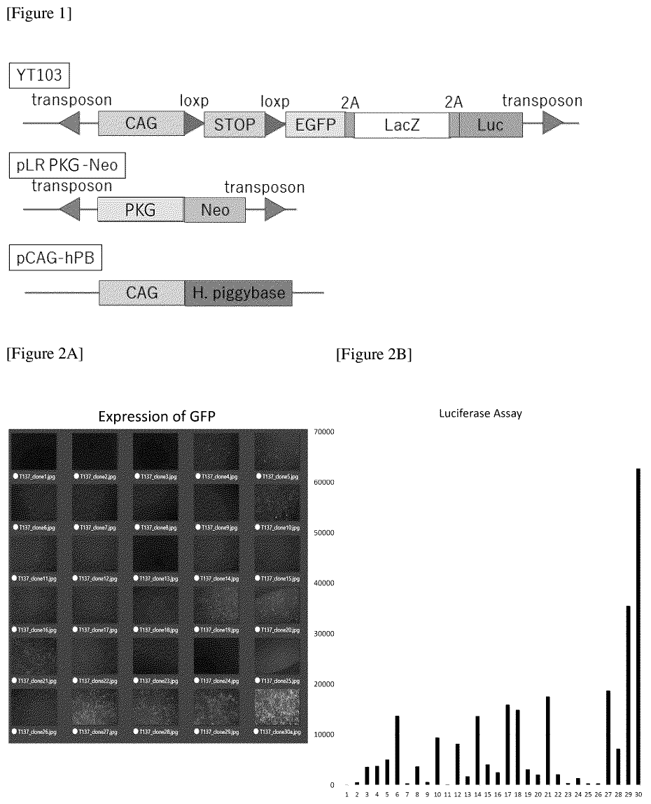

The configuration of the reporter gene expression cassette used in the present Examples is shown in FIG. 1 (YT103). EGFP, LacZ and Luc were used as reporter genes, and a CAG promoter was used as a promoter. A P2A peptide-coding sequence was inserted between individual reporter genes. For the removal of a transcription termination STOP sequence, a Cre/loxP system was used. In addition, the ITR sequence of piggy Bac was used as an ITR sequence of a transposon.

In order to produce the YT103 plasmid (reporter gene expression vector), pLR5-CAG-floxSTOP-EGFP was cleaved with BsrG1. Thereafter, a fragment amplified by PCR with Primer YT198 and Primer YT199, using pAAV-CAG-Luc (Hayashita-Kinoh et al., Mol Ther Methods & Clinical Dev 20:133-141, 2021) as a template; and a fragment amplified by PCR with Primer YT200 and Primer YT201, using pAAV-CAG-LacZ (Ishii et al., Mol Ther Methods & Clinical Dev 18:44-49, 2020) as a template; were inserted therein by employing In-Fusion Cloning Kit (TAKARA).

The primers used in the PCR amplification are as follows.

Primer YT198:
(SEQ ID No: 1)
CTCTCGGCATGGACGAGCTGTACAAGgctactaacttcagcctgctgaag
caggctggagacgtggaggagaaccctggacctGTCGTTTTACAACGTCG
T
Primer YT199:
(SEQ ID No: 2)
GGGTCCTGGATTTTCCTCAACATCTCCACAAGTAAGCAAAGAGCCCCTTC
CTTCTTTTTGACACCAGACCAACTG
Primer YT200:
(SEQ ID No: 3)
TTGAGGAAAATCCAGGACCCatggaagacgccaaaaacat
Primer YT201:
(SEQ ID NO: 4)
AGAGTCGCGGCCGCTTTACTTttacaatttggactttccgc

It is to be noted that the pLR5-CAG-floxSTOP-EGFP plasmid was produce according to the method described in the previous report (Fujita et al., Nat Cell Biol. 22:26-37, 2020). The sequence of the reporter gene expression cassette used in the present Examples is as set forth in SEQ ID No: 5 in the sequence listing.

### 1-2. Selection Marker Expression Vector

The configuration of the selection marker expression cassette used in the present Examples is shown in FIG. 1 (pLR PKG-Neo). A Neo gene was used as a selection marker gene, and a PKG promoter was used as a promoter. In addition, the ITR sequence of piggyBac was used as an ITR sequence of a transposon. The nucleic acid sequence of the selection marker expression cassette used in the present Examples is as set forth in SEQ ID No: 6 in the sequence listing.

The pLR5-PKG-Neo plasmid was produced by cleaving pLR5-CAG-EGFP (Fujita et al., Nat Cell Biol. 22:26-37, 2020) with Sall/BsRGI, and then inserting the sequences amplified by PCR therein employing the Infusion Kit.

### 1-3. Transposase Expression Vector

The configuration of the transposase expression cassette used in the present Examples is shown in FIG. 1 (pCAG-hPB). A piggyBac transposase gene was used as a transposase gene, and a CAG promoter was used as a promoter.

The pCAG-hPB plasmid was produced by cleaving pCAG-EGFP (Tsunekawa et al., The EMBO Journal 31:1879-1891, 2012) with EcoRI/BsRGI, and then inserting the sequences amplified by PCR therein employing the Infusion Kit. The nucleic acid sequence of the transposase gene expression cassette used in the present Examples is as set forth in SEQ ID No: 7 in the sequence listing.

### 2. Preparation of Cells for Use in Detection of AAV-Neutralizing Antibody

Highly sensitive cells exhibiting reporter activity (highly sensitive receiver cells) were screened from among the cells in which a reporter gene expression cassette, a selection marker gene expression cassette, and a transposase gene expression cassette were inserted into the genome thereof (the cells according to the second embodiment).

Using PEI-MAX, a pLR5-CAG-floxSTOP-EGFP plasmid, a pLR5-PKG-Neo plasmid, and a pCAG-hPB plasmid were transfected at a ratio of 20:1:2 (molar ratio) into 293A cells. After the transfection, the cells were cultured in a G418-containing medium for 2 weeks. Thereafter, using BD FACSMelody, the cultured cells were dispensed into a 96-well plate in an amount of 1 cell per well, and were then cultured. After the culture, the cell clones in individual wells were split into a 96-well plate, and were then infected with AAV1-CAG-Cre (an AAV vector for the expression of Cre recombinase). The AAV1-CAG-Cre was prepared by transfecting 3 types of plasmids (pHelper, pR2C1, and pAAV-CAG-Cre) into 293AAV cells, then culturing the resulting cells for 11 days, and then concentrating the obtained culture supernatant using TAKARA AAVpro Concentrator. Three days after the infection, luciferase assay was carried out.

### 3. Selection of Highly Sensitive Cells for Use in Detection of AAV-Neutralizing Antibody (Receiver Cells)

From the obtained 100 clones, T137 cell clone was obtained as a clone from which the strongest signals were obtained according to the luciferase assay performed by the method described in 2 above.

Subsequently, the T137 cells were seeded on a 12-well plate and a 96-well plate at cell densities of about 1×10⁵ cells/well and about 1×10⁴ cells/well, respectively. The seeded cells were cultured in DMEM supplemented with 10% FCS, penicillin/streptomycin, and 200 ng/mL G418, under conditions of 37° C. and 5% CO₂. After the culture for 24 hours, the medium was exchanged with a G418-free medium, and then, AAV1-CAG-Cre was transfected into the cells to result in about 1×10⁵ g.c./cell, and the resulting cells were then cultured under conditions of 37° C. and 5% CO₂.

Four days after the transfection, the expression of GFP in the cells seeded on the 12-well plate was observed under a fluorescence microscope, and fluorescence images were obtained. The medium for the cells seeded on the 96-well plate was removed, and a new medium was added (40 μL/well). Employing Bright-Glo Luciferase Assay System (Promega), luciferase activity was measured using a plate reader.

The fluorescence images of GFP are shown in FIG. 2A. On the other hand, FIG. 2B shows the results of the luciferase assay. From FIG. 2, it was found that T137 clone 30 shows the highest reporter activity. Thus, T137 clone 30 (T137-30) was used in the subsequent experiments.

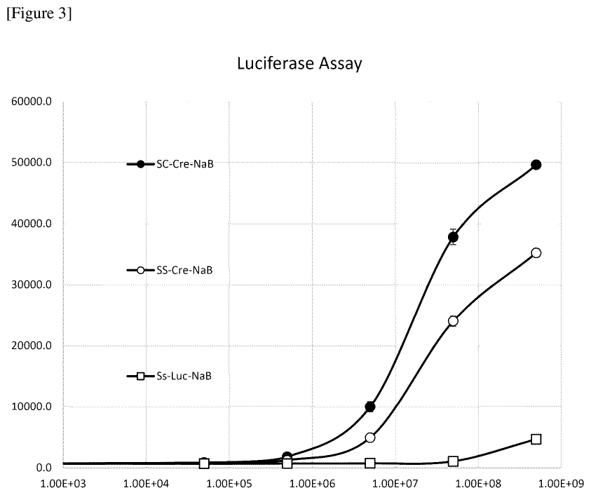

T137-30 cells were seeded in DMEM supplemented with 10% FCS and penicillin/streptomycin, which was on a 96-well plate to result in about 1×10⁴ cells/well. The cells were cultured under conditions of 37° C. and 5% CO₂ for 3 days until the cells became about 90% confluent. After the culture for 3 days, self-replicating Cre packaging AAV1 (SC-Cre), single-stranded Cre packaging AAV1 (SS-Cre), or single-stranded luciferase packaging AAV1 (Ss-Luc) was successively diluted 10-fold from 5×10⁸ gc/well to 5×10⁴ gc/well, in the presence of sodium butyrate, and each diluted AAV1 was transfected into the cells. Each experiment was carried out three times. On the 3rd day after the transfection, the medium was removed from each well, and 40 μL of a new medium was then added thereto. Employing Bright-Glo Luciferase Assay System (Promega), luciferase activity was measured using a plate reader. The results are shown in FIG. 3. When the cells were infected with Ss-Luc used in conventional methods of detecting an AAV-neutralizing antibody, only extremely low luciferase activity was detected. In contrast, when the cells were infected with SC-Cre and SS-Cre, extremely high luciferase activity was detected.

From the aforementioned results, it was demonstrated that when AAV expressing a site-specific recombinant enzyme (Cre recombinase, etc.) is infected into the cells of the present invention in which a reporter gene expression cassette, a selection marker gene expression cassette, and a transposase gene expression cassette have been inserted into the genome thereof, extremely high reporter activity is detected. Therefore, by using this reporter activity detection system, only a trace amount of AAV-neutralizing antibody existing in a sample can be even detected.

### INDUSTRIAL APPLICABILITY

The present invention is a method of detecting the presence or absence and abundance of an AAV-neutralizing antibody in a sample with high sensitivity. Therefore, the present detection method is useful to evaluate the therapeutic effects of diseases using an AAV vector and to evaluate the safety thereof, and utilization of the present detection method in the medical field is expected.

1. A vector composition for detection of an AAV (adeno-associated virus)-neutralizing antibody, the vector composition comprising the following vectors (a), (b) and (c):
(a) a vector, comprising a DNA which is a reporter gene expression cassette in which a 5′ transposon-specific inverted terminal repeat (ITR) sequence (“5′ ITR sequence”), a promoter, a transcription termination STOP sequence that can be removed by a site-specific recombinant enzyme, one or multiple reporter genes, and a 3′ transposon ITR sequence (“3′ ITR sequence”) are connected with one another in this order, the DNA being disposed such that if the transcription termination STOP sequence is removed, the one or multiple reporter genes can be expressed by the promoter,
(b) a vector, comprising a DNA which is a selection marker gene expression cassette in which a 5′ ITR sequence, a promoter, a selection marker gene, and a 3′ ITR sequence are connected with one another in this order, the DNA being disposed such that the selection marker gene can be expressed by the promoter, and
(c) a vector, comprising a DNA which is a transposase gene expression cassette in which a promoter and a transposase gene that recognizes the transposon ITR sequences included in the vectors (a) and (b) are connected with each other in this order, the DNA being disposed such that the transposase gene can be expressed by the promoter.

2. The vector composition according to claim 1, wherein the STOP sequence is disposed between site-specific recombinant enzyme-recognizing sequences disposed in the same direction.

3. The vector composition according to claim 2, wherein the site-specific recombinant enzyme-recognizing sequences are loxP sequences, and the transposon is piggyBac.

4. A cell, in which the reporter gene expression cassette, the selection marker gene expression cassette, and the transposase gene expression cassette according to claim 1 are inserted into the genome thereof.

5. A method of detecting an AAV-neutralizing antibody in a sample, comprising:
adding a mixture of recombinant AAV retaining a site-specific recombinant enzyme gene in an expressible manner and a sample to the cell according to claim 4, so that the AVV is infected into the cell, and
measuring the expression level of a reporter gene.

6. The method according to claim 5, wherein the site-specific recombinant enzyme is Cre recombinase.

7. An AAV-neutralizing antibody detection kit, comprising at least one vector selected from the group consisting of a vector comprising a reporter gene expression cassette, a vector comprising a selection marker gene expression cassette, and a vector comprising a transposase gene expression cassette.

8. The AAV-neutralizing antibody detection kit according to claim 7, comprising:
a cell having the vectors (a), (b) and (c) inserted into the genome thereof:
(a) a vector, comprising a DNA which is a reporter gene expression cassette in which a 5′ transposon-specific inverted terminal repeat (ITR) sequence (“5′ ITR sequence”), a promoter, a transcription termination STOP sequence that can be removed by a site-specific recombinant enzyme, one or multiple reporter genes, and a 3′ transposon ITR sequence (“3′ ITR sequence”) are connected with one another in this order, the DNA being disposed such that if the transcription termination STOP sequence is removed, the one or multiple reporter genes can be expressed by the promoter,
(b) a vector, comprising a DNA which is a selection marker gene expression cassette in which a 5′ ITR sequence, a promoter, a selection marker gene, and a 3′ ITR sequence are connected with one another in this order, the DNA being disposed such that the selection marker gene can be expressed by the promoter, and
(c) a vector, comprising a DNA which is a transposase gene expression cassette in which a promoter and a transposase gene that recognizes the transposon ITR sequences included in the vectors (a) and (b) are connected with each other in this order, the DNA being disposed such that the transposase gene can be expressed by the promoter.

9. A cell, in which the reporter gene expression cassette, the selection marker gene expression cassette, and the transposase gene expression cassette according to claim 2 are inserted into the genome thereof.

10. A method of detecting an AAV-neutralizing antibody in a sample, comprising:
adding a mixture of recombinant AAV retaining a site-specific recombinant enzyme gene in an expressible manner and a sample to the cell according to claim 9, so that the AVV is infected into the cell, and
measuring the expression level of a reporter gene.

11. The method according to claim 10, wherein the site-specific recombinant enzyme is Cre recombinase.

12. The AAV-neutralizing antibody detection kit according to claim 7, comprising
a cell having the vectors (a), (b) and (c) inserted into the genome thereof:
(a) a vector, comprising a DNA which is a reporter gene expression cassette in which a 5′ transposon-specific inverted terminal repeat (ITR) sequence (“5′ ITR sequence”), a promoter, a transcription termination STOP sequence that can be removed by a site-specific recombinant enzyme, one or multiple reporter genes, and a 3′ transposon ITR sequence (“3′ ITR sequence”) are connected with one another in this order, the DNA being disposed such that if the transcription termination STOP sequence is removed, the one or multiple reporter genes can be expressed by the promoter,
(b) a vector, comprising a DNA which is a selection marker gene expression cassette in which a 5′ ITR sequence, a promoter, a selection marker gene, and a 3′ ITR sequence are connected with one another in this order, the DNA being disposed such that the selection marker gene can be expressed by the promoter, and
(c) a vector, comprising a DNA which is a transposase gene expression cassette in which a promoter and a transposase gene that recognizes the transposon ITR sequences included in the vectors (a) and (b) are connected with each other in this order, the DNA being disposed such that the transposase gene can be expressed by the promoter,
wherein the STOP sequence is disposed between site-specific recombinant enzyme-recognizing sequences disposed in the same direction.

13. A cell, in which the reporter gene expression cassette, the selection marker gene expression cassette, and the transposase gene expression cassette according to claim 3 are inserted into the genome thereof.

14. A method of detecting an AAV-neutralizing antibody in a sample, comprising:
adding a mixture of recombinant AAV retaining a site-specific recombinant enzyme gene in an expressible manner and a sample to the cell according to claim 13, so that the AVV is infected into the cell, and
measuring the expression level of a reporter gene.

15. The method according to claim 14, wherein the site-specific recombinant enzyme is Cre recombinase.

16. The AAV-neutralizing antibody detection kit according to claim 7, comprising
a cell having the vectors (a), (b) and (c) inserted into the genome thereof:
(a) a vector, comprising a DNA which is a reporter gene expression cassette in which a 5′ transposon-specific inverted terminal repeat (ITR) sequence (“5′ ITR sequence”), a promoter, a transcription termination STOP sequence that can be removed by a site-specific recombinant enzyme, one or multiple reporter genes, and a 3′ transposon ITR sequence (“3′ ITR sequence”) are connected with one another in this order, the DNA being disposed such that if the transcription termination STOP sequence is removed, the one or multiple reporter genes can be expressed by the promoter,
(b) a vector, comprising a DNA which is a selection marker gene expression cassette in which a 5′ ITR sequence, a promoter, a selection marker gene, and a 3′ ITR sequence are connected with one another in this order, the DNA being disposed such that the selection marker gene can be expressed by the promoter, and
(c) a vector, comprising a DNA which is a transposase gene expression cassette in which a promoter and a transposase gene that recognizes the transposon ITR sequences included in the vectors (a) and (b) are connected with each other in this order, the DNA being disposed such that the transposase gene can be expressed by the promoter,
wherein the STOP sequence is disposed between site-specific recombinant enzyme-recognizing sequences disposed in the same direction, and
wherein the site-specific recombinant enzyme-recognizing sequences are loxP sequences, and the transposon is piggyBac.
